(19) United States
(12) Patent Application Publication
(10) Pub. No.: US 2010/0197012 A1
(43) Pub. Date: Aug. 5, 2010

(54) Application of RNA Interference Targeting dhfr Gene, to Cell for Producing Secretory Protein

(75) Inventors: Suh-Chin Wu, Hsinchu City (TW); Willy W.L. Hong, Longtan Township (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: NATIONAL TSING HUA UNIVERSITY, Hsinchu (TW)

(21) Appl. No.: 12/761,193

(22) Filed: Apr. 15, 2010

Related U.S. Application Data
(63) Continuation-in-part of application No. 11/758,203, filed on Jun. 5, 2007.

(51) Int. Cl. C12N 5/07

(52) U.S. Cl. 435/358

(57) ABSTRACT

Biological materials are applied to a CHO cell or the like for enhancing production of a species of protein. The biological materials includes an expression vector and a silencing vector, the expression vector including a dhfr gene of a species of mammal and a gene encoding the species of protein, the silencing vector including a DNA fragment for inducing a RNA interference in the CHO cell to reduce expressions of both exogenous dhfr gene and endogenous dhfr gene after the biological material is applied to the CHO cell, and the CHO cell is thus not limited to dhfr gene deficient type. The DNA fragment consists of nucleotides characterizing a segment of a dhfr gene of the CHO cell and a segment of a dhfr gene of the species of mammal.

### CROSS REFERENCE TO RELATED APPLICATION

This is a continuation-in-part of prior, pending application Ser. No. 11/758,203, filed Jun. 5, 2007.

### FIELD OF THE INVENTION

The present invention generally relates to application of RNAi (ribonucleic acid interference) to a CHO cell which is not limited to dhfr gene deficient type, for producing (expressing) secretory protein.

### BACKGROUND OF THE INVENTION

Mammalian cells have been extensively utilized to produce recombinant proteins as biopharmaceuticals for clinical applications. Amplifiable selective marker such as dihydrofolate reductase (dhfr), and cells such as those from Chinese hamster ovary (CHO), are routinely used to generate stable producer cell clones. Methotrexate (MTX), a folic acid analog which binds and inhibits DHFR, has been widely used to improve recombinant DNA expression in CHO cells by co-amplifying concurrently a target gene and the DHFR protein therein. Stepwise increasing the concentration of MTX in growth medium can result in hundred to thousand copies of the co-amplified target genes in stable producer CHO cells. To date, several attempts have been made to improve the in vitro selection of stable producer CHO cells through stepwise MTX selection, including an internal ribosome entry site (IRES)-driven dicistronic vector, incomplete splicing (in dhfr and target cDNA) vectors, and the use of less-sensitive mutant dhfr genes to MTX However, in vitro selection of high producer cell clones still remains as the most time-consuming process in CHO cell expression technology, and the conventional technologies to produce recombinant proteins is subject to limited efficiency/stability. The stability problem is particularly significant after ending the application of MTX or in MTX-free medium.

RNA interference (RNAi), initially found in Caenorhabditis elegans, has been considered a natural response to double-stranded RNA for controlling sequence-specific gene expression at a post-transcriptional level. Introducing double-stranded RNA in mammalian cells has emerged as a powerful means to silence gene expression in mammalian cells through RNAi. The double-stranded RNAs, transcribed as short hairpin RNA (shRNA) and processed to be active with a length of 19-23 nucleotides by Dicer, can recognize target mRNAs in a sequence-specific manner. Relevant conventional technologies leave a significant margin for improvement.

Prior arts such as those of Kim et al. (Biotechnology and bioengineering, 1998, 58:73-84), Levinson et al. (U.S. Pat. No. 4,965,196), Yang et al. (US patent 2006/0223144 A1), Xiong et al. (Biotechnology Letters, 2005, 27:1713-1717), Elbashir et al. (Methods, 2002, 26:199-213), etc. do not disclose using a silencing vector in promoting protein production with normal cell as a target cell (i.e., producer cell). Prior arts using producer cells for producing protein are subject to the limitation of using dhfr gene deficient cell as a producer cell (i.e., target cell), because of their lack of mechanism to reduce the expression of endogenous dhfr gene (i.e., the dhfr gene of the target cell). Prior arts using producer cells for producing protein are also subject to the limitation of relying solely on MTX to reduce the expression of exogenous dhfr gene cell.

For more information about relevant RNAi application, reference to inventors' abstract entitled “ENHANCING EXPRESSION OF PROTEIN IN CHO CELLS BY THE CO-AMPLIFICATION OF SPECIFIC shRNA TO DHFR” and posted Jun. 18˜23, 2006 in 20^{th} IUBMB International Congress of Biochemistry and Molecular Biology and 11^{th} FAOBMB Congress, as well as inventors' paper entitled “A novel RNA silencing vector to improve antigene expression and stability in Chinese hamster ovary cells” received Nov. 2, 2006 and to be published in Vaccine Volume 25, Issue 20, May 16, 2007, Pages 4103-4111 by ELSEVIER, shall be made.

### SUMMARY OF THE INVENTION

One of the objects of the present invention is to provide biological materials for applying to at least a cell for promoting production of protein.

Another one of the objects of the present invention is to provide biological materials for applying to at least a target cell for enhancing protein production, with the target cell not limited to a mutated cell (dhfr gene deficient cell), i.e., either a mutated cell or normal cell can be used as the target cell.

A further one of the objects of the present invention is to provide biological materials for applying to at least a cell for upgrading the stability of protein production.

One of the advantages of the present invention is that protein production can be enhanced even without MTX application, and be further enhanced with MTX application.

Another one of the advantages of the present invention is that not only the expression of exogenous DHFR protein (i.e., expression of exogenous dhfr gene) but also the expression of endogenous DHFR protein (i.e., expression of dhfr gene of the cell) can be reduced to achieve production enhancement of protein, thereby the producer cell (target cell) is not limited to dhfr gene deficient type.

Further one of the advantages of the present invention is that higher stability of protein production can be achieved

Another further one of the advantages of the present invention is that high producer clones can be selected while the probability of successful selection is not lowered.

The present invention features application of biological materials to at least a target cell such as a CHO cell, for producing (or expressing, or amplifying) a species of protein such as secretory protein. The biological materials comprise: an expression vector including a first sequence encoding DHFR protein of a species of mammal (e.g. a mouse) and a second sequence encoding the species of protein to be produced (or expressed, or amplified); and a silencing vector including a DNA (deoxyribonucleic acid) fragment for inducing, after the biological material is applied to the target cell, a RNA interference in the target cell to reduce (i.e., to silence or inhibit or knock down) not only the expression of exogenous DHFR protein (i.e., the expression of the dhfr gene of the species of mammal), but also the expression of endogenous DHFR protein (i.e., the expression of the dhfr gene of the target cell), thereby the production (or expression, or amplification) of the protein is enhanced regardless of whether or not the target cell is a dhfr gene deficient cell. After the expression vector and the silencing vector are co-transfected (i.e., are mixed to be the biological material and then be transfected) to the target cell, the DNA fragment of the silencing vector induces a RNA interference in the target cell to reduce both the expression of exogenous DHFR protein (the expression of the dhfr gene of the species of mammal), but also the expression of endogenous DHFR protein (the expression of the dhfr gene of the target cell), thereby the production (or expression, or amplification) of the protein is enhanced regardless of whether or not the target cell is a dhfr gene deficient cell.

Alternatively the silencing vector in the biological material provided according to the present invention includes a DNA fragment for inducing, after the biological material is applied to (i.e., transfected to) the target cell, a RNA interference in the target cell to reduce (i.e., to silence or inhibit or knock down) expression of a gene of relevance in the target cell. The first sequence included in the expression vector according to the present invention is usually in the same species as the gene of relevance. For example, if the gene of relevance with expression to be reduced in the target cell is a dhfr (dihydrofolate reductase) gene, the first sequence included in the expression vector is usually a dhfr gene sequence. The gene of relevance is usually able to resist, at least partially, the chemical material exogenous to the target cell. The selection of the target cell is not limited to a mutated cell (e.g. a dhfr gene deficient cell), i.e., a mutated cell or normal cell or cell line can be selected as the target cell. Specific example of the protein to be produced is secretory protein (e.g. antibody), and the species of the protein to be produced is not necessarily limited to secretory protein. The gene of relevance comprises (or is constituted at least partially by), either the first sequence included in the expression vector or the gene which is endogenous to the target cell and is in the same species as the first sequence included in the expression vector. The gene of relevance may also comprise (or be constituted at least partially by) both the first sequence included in the expression vector and the gene which is endogenous to the target cell and is in the same species as the first sequence included in the expression vector.

After applying the biological material to a target cell, a composition forms to comprise: the first sequence encoding a DHFR protein of a species of mammal (e.g. a mouse), the second sequence encoding the species of protein to be produced (or expressed, or amplified), and at least a double stranded RNA transcripted in the target cell by the DNA fragment, in addition to the target cell. The composition may be seen as a target cell to which the expression and the silencing vectors have been applied (e.g. co-transfected), i.e., seen as a target cell including the first sequence, the second sequence, and at least a double stranded RNA transcripted therein by the DNA fragment included in the silencing vector.

For an aspect of the present invention in which the expression vector comprises a gene of a species of mammal (e.g. mouse) and a gene encoding the species of protein (e.g. secretory protein), the selection of the DNA fragment in the silencing vector, for example, is such that the DNA fragment consists of nucleotides characterizing a segment of a gene of the target cell and a segment of the gene of the species of mammal. Preferably the gene of the species of mammal is in the same species as the gene of the target cell; for example, the nucleotides in the DNA fragment characterize a segment of a DHFR protein of the target cell and a segment of a DHFR protein of the species of mammal. Specifically, the DNA fragment consists of nucleotides which are respectively equivalent to at least part of the nucleotides in the segment of the gene of the target cell, and are respectively equivalent to at least part of the nucleotides in the segment of the gene of the species of mammal included in the expression vector. For example, the DNA fragment is designed to consist of a plurality of nucleotides which are respectively equivalent to at least part of the nucleotides in a segment of a DHFR protein of the target cell, and are respectively equivalent to at least part of the nucleotides in a segment of a DHFR protein of the species of mammal included in the expression vector. Preferably, if an arbitrary nucleotide in the DNA fragment is equivalent to a nucleotide which is in the segment of the gene of the target cell, the arbitrary nucleotide in the DNA fragment is also equivalent to a nucleotide which is in the segment of the gene of the species of mammal and has the same nucleotide sequence as the arbitrary nucleotide. Preferably, the segment or the DNA fragment has a length of at least 19 nucleotides.

For the aspect of the present invention in which the expression vector comprises a gene of a species of mammal and a gene of the species of protein, the gene of the species of mammal included in the expression vector can be from a mammal which is in the same species as the target cell or in a species different from the species of the target cell.

For a specific aspect of the present invention in which the target cell is a CHO (Chinese hamster ovary) cell and the expression vector comprises a sequence encoding DHFR protein of mammal (such as gi:68299777 of mouse) and a sequence encoding the species of protein, the selection of the DNA fragment in the silencing vector, is such that the DNA fragment consists of nucleotides equal to those which are in a segment of a dhfr gene (such as gi:191045 or SEQ ID NO: 2) of a CHO cell and are in a segment of the dhfr gene (such as SEQ ID NO: 1) of a mouse. The segment of the dhfr gene of a CHO cell and the segment of the dhfr gene of a mouse, preferably both consist of nucleotides with sequence numbers ranging from 99 to 117 (marked with sd2 as shown in FIG. 1A, i.e., SEQ ID NO: 4), or from 187 to 205 (marked with sd3 as shown in FIG. 1A, i.e., SEQ ID NO: 5), or from 413 to 431 (marked with sd1 as shown in FIG. 1A, i.e., SEQ ID NO: 3), resulting in the fact that an arbitrary nucleotide in the segment of the dhfr gene of a mouse is equal to a nucleotide which is in the segment of the dhfr gene of a CHO cell and has the same sequence number (in the dhfr gene of the CHO cell) as the arbitrary nucleotide has (in the dhfr gene of the mouse). For example, as shown in FIG. 1A, all the nucleotides of segment sd2 (SEQ ID NO: 4) are common to both the dhfr gene of a mouse (SEQ ID NO: 1) and the dhfr gene of a CHO cell (SEQ ID NO: 2), with sequence numbers ranging from 99 to 117 in both the dhfr gene of a mouse and the dhfr gene of a CHO cell.

In this disclosure, “CHO cell” means “normal CHO cell” or “dhfr gene deficient CHO cell” or “normal CHO cell and dhfr gene deficient CHO cell together; “CHO/dhfr-” means “dhfr gene deficient CHO cell” and “CHO-k1” means “normal CHO cell”; “pDHFR/EGFP” represents “reporting vector made of dhfr/egfp fusion”, “pEGFP-DHFR” represents “an expression vector including a sequence encoding EGFP protein and another sequence encoding DHFR protein”, and “MTX application” represents “conventionally used adaptable algorithm of applying MTX to production or expression of desired protein based on cells”.

### Contrast of the Present Invention to Conventional Technologies

Prior arts with CHO cell as target cells (i.e., producer cells) for producing protein is limited to using dhfr gene deficient type of CHO cell. The limitation on prior arts results from its lack of effective mechanism in reducing expression of endogenous DHFR protein (i.e., expression of dhfr gene of the target cell). In contrast, the present invention with CHO cell as target cells for producing protein is not limited to using dhfr gene deficient type of CHO cell, i.e., the CHO cell used by the present invention for producing protein can be of any type. The freedom provided by the present invention to use a CHO cell regardless of the cell type, results from an important feature of the silencing vector provided by the present invention. The important feature of the silencing vector provided by the present invention is that it includes a DNA (deoxyribonucleic acid) fragment for inducing (after the biological material including an expression vector and the silencing vector provided by the present invention is applied to the CHO cell), a RNA interference in the CHO cell to reduce not only the expression of exogenous DHFR protein (i.e., the expression of dhfr gene of the species of mammal included in the expression vector), but also the expression of endogenous DHFR protein (i.e., the expression of dhfr gene of the CHO cell), thereby CHO cell clones with higher and more stable expression can be selected, and the production (or expression, or amplification) of the protein encoded by the second sequence included in the expression vector can be enhanced regardless of the type of the CHO cell used as a target cell. In addition to the important feature, the other features of the present invention are recited as follows.

Another feature of the present invention in contrast with prior arts characterizing no-use of silencing vector for producing protein, is that the protein production according to the present invention can be enhanced even without MTX application, as can be seen from the experimental result shown in FIG. 10. In FIG. 10, R:S=1:0 means the amount ratio of applied expression vector to applied silencing vector is 1:0, and R:S=1:1 means the amount ratio of applied expression vector to applied silencing vector is 1:1, where the expression vector includes a first sequence encoding a DHFR protein of mouse and a second sequence encoding a secretory protein (e.g. IgG1-E3.3), and the silencing vector characterizes sd2 (SEQ ID NO: 4). The first two digits of the numeric symbols 10-4, . . . , 10-16, 11-7, . . . , and 11-14, represent the amount ratio of applied expression vector to applied silencing vector, and the number following the first two digits represent the ID of a tested cell (dhfr gene deficient CHO cell). As can be seen from FIG. 10, with CHO/dhfr- (i.e., dhfr gene deficient CHO cell) as the target cell, and with MTX=1 μM (i.e., MTX concentration=1 μM), the expression of the secretory protein IgG1-E3.3 with R:S=1:1 (i.e., the amount ratio of applied expression vector to applied silencing vector is 1:1) is significantly much higher than that with R:S=1:0 (i.e., no silencing vector provided according to the present invention is applied). Even with MTX=0 μM, the expression of the secretory protein IgG1-E3.3 with R:S=1:1 (i.e., the amount ratio of applied expression vector to applied silencing vector is 1:1) is much higher than that with R:S=1:0 (i.e., without application of silencing vector). When mass production is embodied, the difference between R:S=1:1 and R:S=1:0 can be very significant even with MTX=0 μM. With CHO-K1 (i.e., normal CHO cell) as the target cell, and with MTX=1 μM (i.e., MTX concentration=1 μM) or MTX=0 μM, the expression of the secretory protein IgG1-E3.3 with R:S=1:1 (i.e., the amount ratio of applied expression vector to applied silencing vector is 1:1) is commensurable to that with CHO/dhfr- (i.e., dhfr gene deficient CHO cell) as the target cell, i.e., the expression of the secretory protein IgG1-E3.3 with R:S=1:1 (i.e., the amount ratio of applied expression vector to applied silencing vector is 1:1) and with normal CHO cell as the target cell is significantly much higher than that without application of silencing vector, even with MTX=0 μM.

Prior arts such as those of Kim et al. (Biotechnology and bioengineering, 1998, 58:73-84), Levinson et al. (U.S. Pat. No. 4,965,196), Yang et al. (US patent 2006/0223144 A1), Xiong et al. (Biotechnology Letters, 2005, 27:1713-1717), etc. do not use a silencing vector in promoting protein production with normal cell as a target cell (i.e., producer cell). Prior arts are subject to the limitation of using dhfr gene deficient cell as a target cell for producing protein, because of their lack of mechanism to reduce the expression of endogenous dhfr gene (i.e., the dhfr gene of the target cell). In contrast, the silencing vector provided according to the present invention is capable of reducing both the expression of exogenous dhfr gene (i.e., the expression of the DHFR protein of mouse) and the expression of endogenous dhfr gene (i.e., the dhfr gene of the target cell), and thereby the target cell used for producing protein according to the present invention is not limited to dhfr gene deficient type, i.e., either a normal CHO cell or a CHO cell of dhfr gene deficient type can be used as a target cell for producing protein according to the present invention. In addition to the freedom of choosing any type of CHO cells as target cells, the present invention, even without using MTX, can enhance production of protein by the silencing vector capable of reducing both the expression of exogenous dhfr gene and the expression of endogenous dhfr gene (i.e., the dhfr gene of the target cell). The production of protein according to the present invention can be further enhanced with application of MTX.

Some prior arts deemed to possibly relate to protein production with CHO cells as target cells (i.e., producer cells or host cells) are recited as follows.

Kim et al. (Biotechnology and bioengineering, 1998, 58:73-84) use a two-vector system to express antibody with dhfr-deficient CHO cells (DG44) as host cells (target cells). One vector expresses DHFR protein and the light chain of the antibody, and the other vector expresses the heavy chain of the antibody. High-producer subclones are established through the procedure of MTX-based and dhfr-mediated gene amplification-based selection. Kim et al. are subject to the limitation of using dhfr-deficient CHO cells as target cells. Kim et al. are also subject to the limitation of relying solely on MTX application. No silencing vector or the like for enhancing antibody production is disclosed by Kim et al.

Levinson et al. (U.S. Pat. No. 4,965,196) construct a polycistronic expression vector encoding DHFR protein and a desired protein (e.g., HBsAg), to promote production of the desired protein with dhfr-deficient CHO cells as target cells. MTX is employed to induce dhfr-mediated gene amplification for promoting production of the desired protein. Levinson et al. are subject to the limitation of using dhfr-deficient CHO cells as target cells. Levinson et al. are also subject to the limitation of relying solely on MTX application. No silencing vector or the like for enhancing production of the desired protein is disclosed by Levinson et al.

Yang et al. (US patent 2006/0223144 A1) construct an expression vector encoding DHFR protein of mouse and a desired protein (e.g., FSH) for mass production of the desired protein with dhfr-deficient CHO cells as target cells. The expression vector according to Yang et al. is equivalent to the expression vector according to the present invention, but Yang et al. rely solely on MTX application to reduce the expression of exogenous dhfr gene (i.e., expression of DHFR protein of mouse) in promoting production of the desired protein. Yang et al. are subject to the limitation of using dhfr-deficient CHO cells as target cells. Yang et al. are also subject to the limitation of relying solely on MTX application. No silencing vector or the like for enhancing production of the desired protein is disclosed by Yang et al.

Xiong et al. (Biotechnology Letters, 2005, 27:1713-1717) use CHO/dhfr- cells to establish cell clones for producing chimeric antibodies. Expression of dhfr gene included in its expression vector is weakened by incomplete splicing so that production of the chimeric antibodies may be promoted through amplification in MTX after the expression vector is applied to target cells (CHO/dhfr- cells). There is no mechanism to reduce expression of endogenous dhfr gene according to Xiong et al., and Xiong et al. are subject to the limitation of using dhfr-deficient CHO cells as target cells. In contrast, not only the expression of exogenous dhfr gene (i.e., the expression of DHFR protein of mouse) but also the expression of endogenous dhfr gene (i.e., the expression of the dhfr gene of target cells) is reduced according to the present invention, and the present invention is free to use any type of CHO cells as target cells. Expression of dhfr gene is reduced by triggering degradation of mRNA of the dhfr gene according to the present invention. Xiong et al. are also subject to the limitation that an expression vector in use has to be altered in a specific way based on what is suggested by Xiong et al. In contrast, the silencing vector provided according to the present invention can be used with different expression vectors (without need of alteration) as long as expression of exogenous dhfr gene and/or endogenous dhfr gene can be reduced.

Elbashir et al. (Methods, 2002, 26:199-213) provide a protocol for selecting siRNA sequences. Elbashir et al. suggest selecting the target region from 50-100 nucleotides downstream of the start codon of “a given cDNA sequence” and searching for sequences 5′-AA(N19)UU or 5′-AA(N21)UU comprising about 32-79% G/C content. Elbashir et al. establish the rules of selecting siRNA, but do not disclose exactly which region can be a target of siRNA, much less a silencing vector to reduce expression of dhfr gene for promoting production of desired protein, and further much less a silencing vector to reduce expressions of both exogenous dhfr gene and endogenous gene in producing a desired protein.

With resultant protein expression significantly higher than conventional technologies characterizing no-use of the silencing vector provided according to the present invention, the application of silencing vector according to the present invention, however, does not affect cell survival rate, and may even slightly or significantly increase cell survival rate, as can be seen from FIG. 5 (with CHO/dhfr- cells, i.e., dhfr gene deficient CHO cells, as target cells) and FIG. 7 (with normal CHO cells as target cells). In FIGS. 5 and 7, each vertical axis represents cell survival rate (%), each horizontal axis represents concentration (μM) of MTX, the line with marks ♦ or ⋄ represents the case with R:S=1:0 (i.e., the amount ratio of applied expression vector to applied silencing vector is 1:0), the line with marks ▪ or □ represents the case with R:S=1:1 (i.e., the amount ratio of applied expression vector to applied silencing vector is 1:1), and the line with marks ▴ or Δ represents the case with R:S=1:5 (i.e., the amount ratio of applied expression vector to applied silencing vector is 1:5).

Another aspect of contrast of the present invention to those representing conventional technologies and characterizing no-use of the silencing vector provided according to the present invention, is typically shown by FIG. 9. In FIG. 9, the left part and the right part are respectively for illustrating the cases with stable producer cell clones (respectively obtained from dhfr gene deficient CHO/dhfr- cells and normal CHO-K1 cells) grown in MTX-free medium for two weeks, where the effect of silencing vectors on the stability of EGFP protein expression represents the effect of silencing vectors on the stability of protein production. In FIG. 9, the left vertical axis means the relative expression of reporter gene, and the right vertical axis means the percentage to the original expression amount (MTX=5 μM) for each clone; R:S=1:0 means the amount ratio of applied expression vector to applied silencing vector is 1:0, R:S=1:1 means the amount ratio of applied expression vector to applied silencing vector is 1:1, and R:S=1:5 means the amount ratio of applied expression vector to applied silencing vector is 1:5; the first two digits of the numeric symbols 10-2, . . . , 11-16, . . . , 15-16, etc represent the amount ratio of applied expression vector to applied silencing vector, and the number following the first two digits represent the ID of a tested cell (or surviving clone).

It can be seen from FIG. 9 that the stability of EGFP protein production with application of silencing vector (corresponding to the silencing vector in the context of the disclosure) according to the present invention can be significantly higher (almost twice) than conventional technologies characterizing no-use of silencing vector.

More about the FIGS. 5-9 will be seen from relevant description below.

Other advantages, objects, and features of the present invention may be seen from the following detailed description with reference to the drawings.

The present invention may best be understood through the following description with reference to the accompanying drawings, in which:

### BRIEF DESCRIPTION OF THE DRAWINGS

FIG. 1A shows nucleotide sequence of dhfr gene (i.e., sequence encoding DHFR protein) of mouse (SEQ ID NO: 1), and nucleotide sequence of dhfr gene (i.e., sequence encoding DHFR protein) of Chinese hamster (SEQ ID NO: 2), wherein “-” represents the nucleotide the same as the one above, i.e., the same as the one with the same sequence number in the sequence encoding DHFR protein of mouse, and wherein three segments respectively marked with sd1 (SEQ ID NO: 3), sd2 (SEQ ID NO: 4), and sd3 (SEQ ID NO: 5), are to be respectively adopted for designing the DNA fragment included in the silencing vector according to the present invention.

FIG. 1B shows double stranded RNAs respectively represented by SEQ ID NO: 6, SEQ ID NO: 7, and SEQ ID NO: 8, and respectively corresponding to the gene segments sd1, sd2, and sd3 of FIG. 1A, i.e., the one marked with SEQ ID NO: 6 is designed according to sd1, the one marked with SEQ ID NO: 7 is designed according to sd2, and the one marked with SEQ ID NO: 8 is designed according to sd3.

FIG. 2 is a diagram for illustrating a reporting vector and an expression vector.

FIG. 3 is for illustrating the selection of a silencing vector.

FIGS. 4A and 4B are for illustrating the ratio of DHFR expression to GAPDH expression versus different amount of silencing vector applied to a target cell according to the present invention.

FIG. 5 is for illustrating the survival rate of CHO/dhfr- cells after different amount of silencing vectors are applied according to the present invention.

FIG. 6 is for illustrating protein expression in CHO/dhfr- cells for different amount of silencing vectors applied thereto and for the application of different amount of MTX.

FIG. 7 is for illustrating the survival rate of CHO-K1 cells (i.e., normal CHO cells) after different amount of silencing vectors are applied according to the present invention.

FIG. 8 is for illustrating protein expression in CHO-K1 cells (i.e., normal CHO cells) for different amount of silencing vectors applied thereto and for the application of different amount of MTX.

FIG. 9 is for illustrating stability of CHO cells for different amount of silencing vectors applied thereto.

FIG. 10 is for illustrating IgG secretion of stable clones for production of secretory protein.

FIG. 11 is for illustrating stability of secretory IgG produced in CHO cells.

### DETAILED DESCRIPTION OF THE PREFERRED EMBODIMENTS/PRESENT INVENTION

### Materials and Methods

### Construction of Plasmids (i.e., Vectors)

To evaluate the effect of reducing expression of dhfr gene by the silencing vector provided according to the present invention, reporting vector (pDHFR/EGFP) of dhfr/egfp fusion is used to be co-transfected with the silencing vector in CHO cells (i.e., is mixed with the silencing vector and transfected in CHO cells). Expression of EGFP protein is much easier to be detected than that of DHFR protein (dhfr gene), the effect of reducing expression of DHFR protein (i.e., expression of dhfr gene) by using the silencing vector is thus much easier to be measured by detecting the expression of the reporting vector (pDHFR/EGFP) of dhfr/egfp fusion. The reporting vector (pDHFR/EGFP) of dhfr/egfp fusion is constructed by inserting dhfr/egfp fusion sequence including mouse dhfr cDNA followed by egfp sequence, into BamHI and EcoRI sites of pcDNA3.1(+) driven by cytomegalovirus (CMV) immediate-early gene promoter and enhancer.

To evaluate the effect of enhancing production of EGFP protein by the silencing vector provided according to the present invention, expression vector (pEGFP-DHFR) is used to be co-transfected with the silencing vector in CHO cells (i.e., is mixed with the silencing vector and transfected in CHO cells). The expression vector (pEGFP-DHFR) is generated by replacing the neomycin phosphotransferase gene (neo) gene with mouse dhfr cDNA driven by an SV40 promoter in the pcDNA3.1(+) (Invitrogen). The EGFP fragment from pEGFP-N1 (BD Biosciences), driven by cytomegalovirus (CMV) immediate-early gene promoter and enhancer, is cloned into BamHI and EcoRI sites. The internal ribosomal entry site (IRES) on pIRES (BD Biosciences) and the Zeocin (Zeo) gene from pcDNA3.1/Zeo (Invitrogen) are cloned into XhoI/XbaI and XbaI/ApaI sites, respectively, to select colonies.

To evaluate the effect of enhancing production of secretory protein by the silencing vector provided according to the present invention, expression vector (IgG-expression vector) including a first sequence encoding DHFR protein of mouse and a second sequence encoding secretory protein such as antibody protein, is used to be co-transfected with the silencing vector in CHO cells (i.e., is mixed with the silencing vector and transfected in CHO cells). The expression vector (IgG-expression vector) for chimeric full-length IgG1 expression, is constructed from plasmid pFab CMV-dhfr 2H7, including a dual expression cassette with CMV promoters and poly(A) site for the heavy chain and light chain of IgG, mouse DHFR expression cassette, and neo and lactamase (amp) genes. The DNA fragments of variable regions of heavy chain (V_{H}) and light chain (V_{L}) from Japanese encephalitis virus-neutralizing antibody, E3.3, are used to replace those in the pFab CMV-dhfr 2H7 and generate a chimeric whole IgG1 expression vector.

To evaluate the effect of enhancing production of desired protein by three different silencing vectors provided according to the present invention, three silencing vectors respectively including DNA fragment consists of nucleotides characterizing sd1 (i.e., SEQ ID NO: 3), sd2 (i.e., SEQ ID NO: 4), and sd3 (i.e., SEQ ID NO: 5) are constructed. Human polymerase-III U6 promoter is amplified from the genomic DNA of HeLa cells (ATCC, CCL-2) and cloned in front of the mouse/hamster dhfr-specific shRNA with five thymidines as a terminator signal to construct shRNA silencing vectors (pCMVen-sd1, pCMVen-sd2, and pCMVen-sd3). The CMV promoter and the enhancer are replaced with a CMV enhancer upstream of the U6 promoter to increase the efficacy of silencing in the identification of shRNA candidates. The control vector (pCMVen) has the same CMV enhancer/U6 promoter with no shRNA.

### Cell Culture and Transfection

CHO-K1 cells (Normal CHO cells) (ATCC, CCL-61) and the dhfr-deficient CHO/dhFr- cells (ATCC, CRL-9096), to be used as target cells, are obtained from FIRDI (Taiwan) and cultured at 37° C. in a humidified incubator with 5% CO₂. CHO-K1 and CHO/dhfr- cells, before transfection and cloning selection, were maintained in Ham's F-12K and MEM-α media supplemented with 10% fetal bovine serum (FBS) (Invitrogen), 100 units/ml penicillin, and 100 μg/ml streptomycin (Invitrogen). CHO cells were transfected with expression and silencing vectors using Lipofectamine 2000 (Invitrogen).

### Western Blotting

CHO cells that had been transfected with reporting and silencing vectors were harvested. Equal amounts of protein were resolved by SDS-PAGE (12% polyacrylamide) and transferred to a nitrocellulose membrane (Millopore) by electroblotting Immune complexes were visualized with BCIP/NBT substrate kit (Invitrogen) using anti-DHFR IgG (BD Biosciences), anti-GAPDH IgG (Abcam) and Anti-mouse IgG-AP.

### Stable Clone Selection and Gene Amplification

The CMV enhancer was removed before transfection to prevent interference by the CMV enhancer of CMV enhancer-promoter-dependent transcription. The linearized plasmids (2 μg) were co-transfected into CHO-K1 and CHO/dhfr- cells with a 1:0, 1:1 or 1:5 ratio of the expression vector (pEGFP-DHFR) to the psd2 silencing vector that includes only CMV promoter without the enhancer. Following transfection, the cells were transferred into two 60 mm plates, grown for two days in a nonselective growth medium, and then replaced the medium with ribonucleosite/deoxyribonucleosite-free MEM-α (Invitrogen) with 10% dialyzed serum (DS, Invitrogen), 200 μg/ml Zeocin (Invitrogen) and 600 μg/ml G-418 (Calbiochem) to select DHFR-, Neo-, and Zeo-positive colonies. Only Zeocin was used to select colonies of the control transfected cells (pEGFP-DHFR only). Ten to 14 days after the cells were transferred to the selective medium, a limiting dilution in the 96-well plates was employed to isolate cell clones and the wells that contain single cells are labeled under fluorescent microscopy. Single cell clones are isolated and 18 of the colonies that expressed the highest level of EGFP or IgG are employed in the subsequent methotrexate (MTX)-driven amplification. The MTX concentration in CHO cell culture medium is increased from 0.04 μM to 1 μM (IgG-expressed clones) or 5 μM (EGFP expressed clones). In each selection step, cells are cultivated for at least 15 days before the MTX concentration is increased.

### Real-Time PCR and RT-PCR

The genomic DNA extraction from stable CHO cell clones was performed using DNeasy Tissue kit (Qiagen). The RNAs were extracted from the transfected cells using Trizol (Invitrogen). The primers and Taqman probe (Applied Biosystems assay ID 293340) were employed for real-time PCR measurement to determine the copy numbers of mouse dhfr gene. One-step RT-PCR was performed to obtain the amount of RNA transcripts of mouse and hamster dhfr genes. The real-time PCR and RT-PCR assays were performed on an ABI PRISM 7500 real time PCR system, by calculating the absolute amount of DNA and the relative amount of RNA by the ΔΔCt method, while the amounts of RNA transcripts were normalized to that of eukaryotic 18s rRNA.

### Enzyme-Linked Immunosorbent Assay (ELISA)

IgG concentration in culture supernatants was determined according to ELISA. Specific productions were determined at 72 h post cell inoculation. Plates (Coring, 9018) were pre-coated with domain III of JEV envelop proteins and the bound antibodies quantified with an anti-human-Fc antibody-HRP and TMB substrate (Pierce). To calculate the specific productions, the total IgG produced was divided by the integral of the total cell number and 3 (3 days). Results are given as pg/cell/day.

### Results

### Silencing Vector Targeting DHFR Protein Expression in CHO/dhfr-Cells

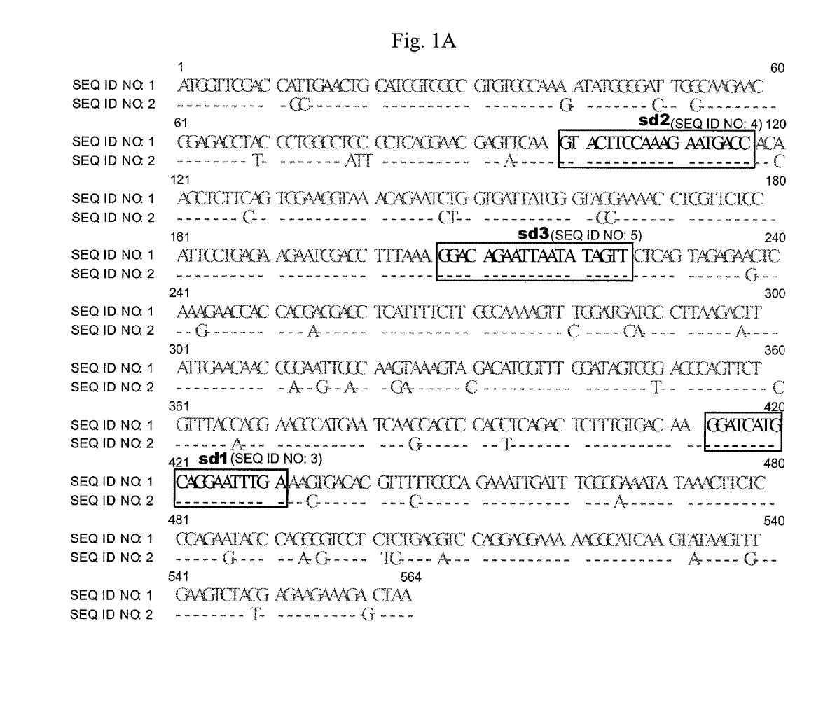

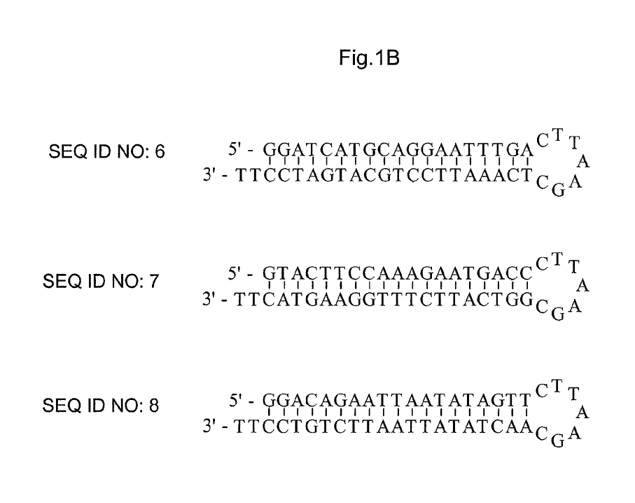

Three sequence segments GT ACTTCCAAAG AATGACC (SEQ ID NO: 4, marked with sd2 in FIG. 1A), GGAC AGAATTAATA TAGTT (SEQ ID NO: 5, marked with sd3 in FIG. 1A), and GGATCATG CAGGAATTTG A (SEQ ID NO: 3, marked with sd1 in FIG. 1A), located in the conserved sequences of the mouse and Chinese hamster DHFR proteins, as shown in FIG. 1A, were respectively selected for constructing three different silencing vectors, to silence or reduce or inhibit or knock down exogenous (mouse) and/or endogenous (Chinese hamster) DHFR proteins in CHO/dhfr- and CHO-K1 cells. The three silencing vectors, respectively representing a different one of three shRNAs which respectively correspond to the sd1 (SEQ ID NO: 3), sd2 (SEQ ID NO: 4), and sd3 (SEQ ID NO: 5), and driven by human polymerase-III U6 promoter and the CMV enhancer, were constructed (as shown in FIG. 1B).

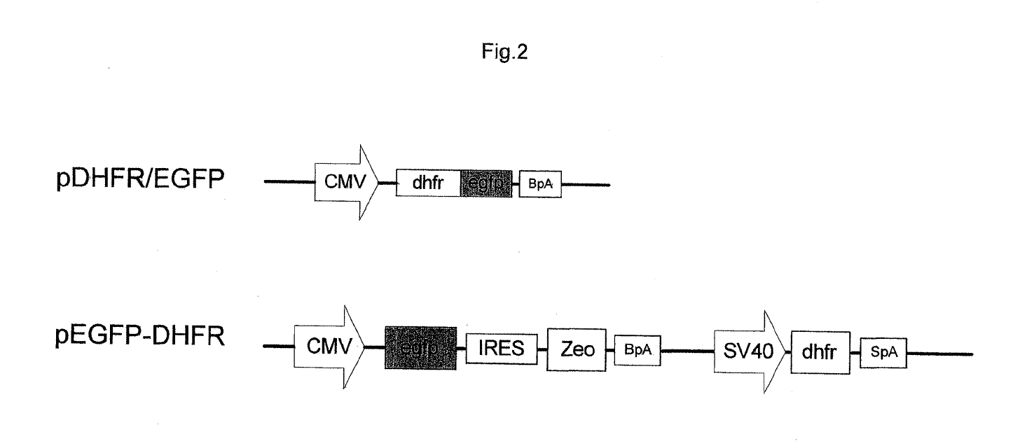

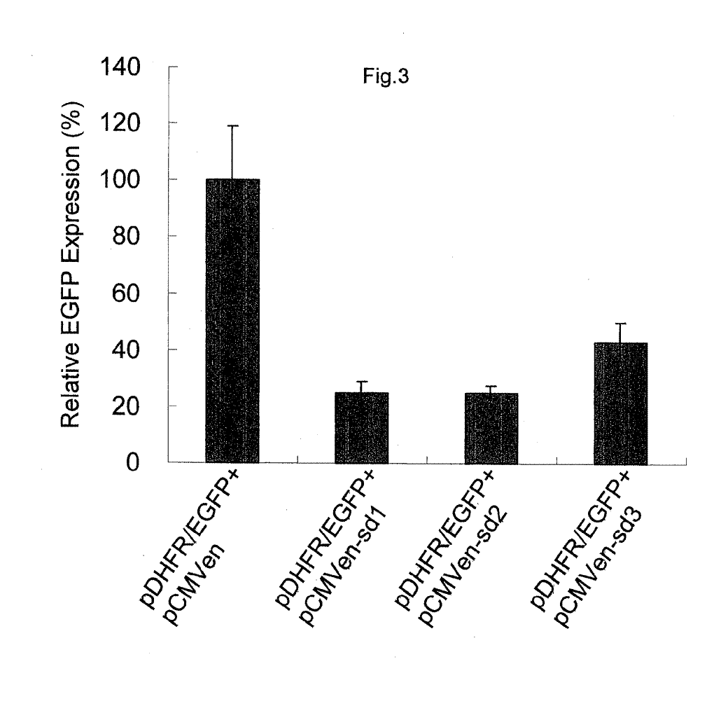

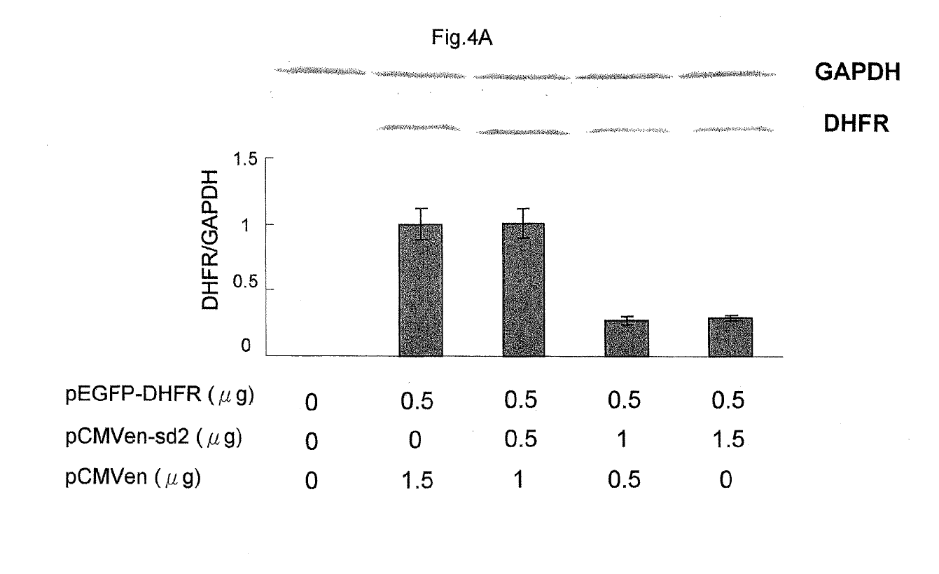

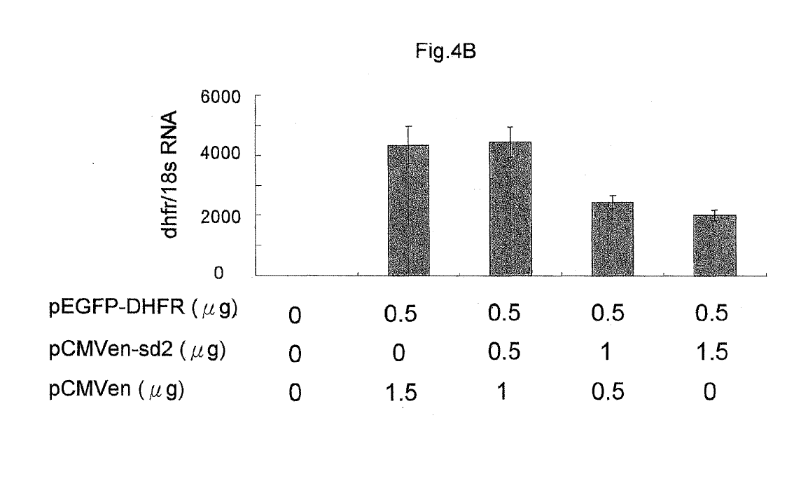

To evaluate the effects of dhfr-silencing, reporting vector (pDHFR/EGFP) encoding DHFR/EGFP fusion protein is constructed (FIG. 2), and silencing vectors pCMVen-sd1, pCMVen-sd2 and pCMVen-sd3 are respectively co-transfected with the reporting vector pDHFR/EGFP in CHO/dhfr- cells, the effects of dhfr-silencing can be detected by measuring the reduction of EGFP expression because DHFR protein and EGFP protein have been fused together as a unit of fusion. As can be seen from FIG. 3, application of the silencing vectors results in the reduction of EGFP expression levels by 74% (pCMVen-sd1), 75% (pCMVen-sd2) and 57% (pCMVen-sd3) as determined by using Wallac Victor² Multicounter (PerkinElmer Life Science). Dose dependent experiments showed that 1 μg and 1.5 μg of the silencing vector pCMVen-sd2 resulted in a maximum of reducing EGFP expression by over 70% (normalized to GAPDH) (as shown in FIG. 4A), correlated with the reduction of dhfr RNA transcripts by around 60% (normalized to 18 sRNA), as shown in FIG. 4B. The silencing vector pCMVen-sd2 was demonstrated to be most effective to silence dhfr RNA transcripts and knockdown EGFP gene expression under the conditions tested.

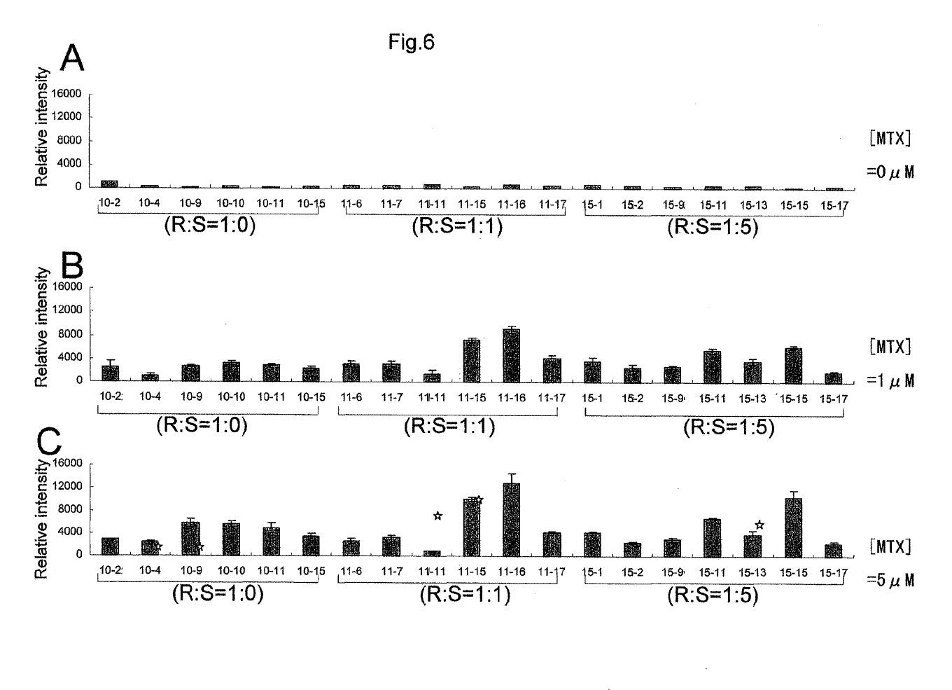

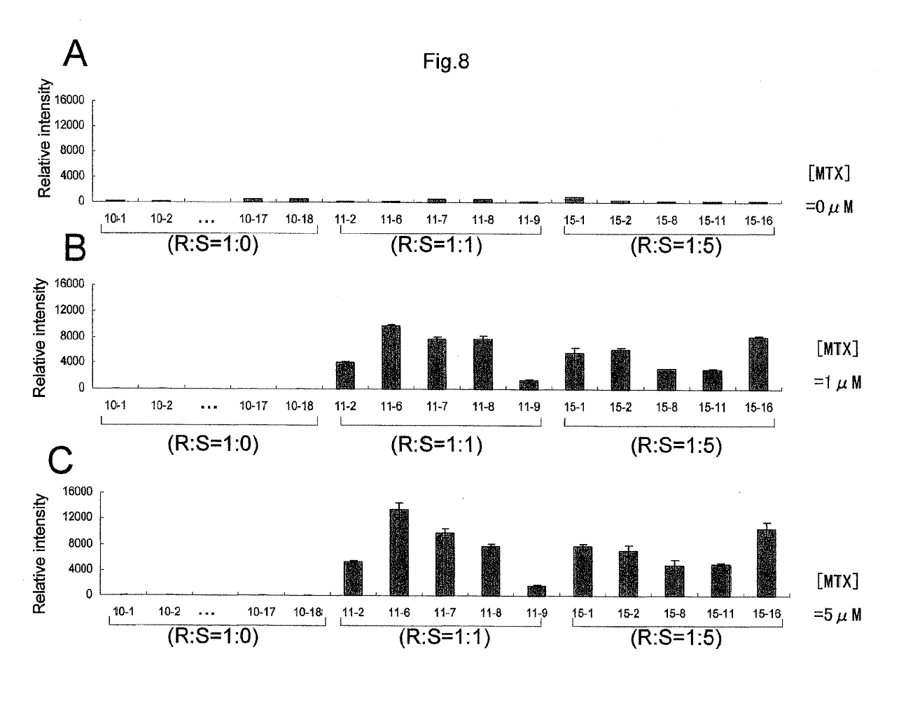

To evaluate the effect of enhancing production of EGFP protein by the silencing vector pCMVen-sd2, expression vector (pEGFP-DHFR) encoding the EGFP and DHFR proteins (preferably under two different promoters) are constructed (FIG. 2) and co-transfected with the silencing vector pCMVen-sd2 in CHO/dhfr- cells or normal CHO cells, i.e., the expression vector is mixed with the silencing vector to form a biological material and the biological material is applied to the CHO/dhfr- cells or normal CHO cells. The results are typically shown by FIG. 6 (with CHO/dhfr- cells, i.e., dhfr gene deficient CHO cells, as target cells) and FIG. 8 (with normal CHO cells, i.e., dhfr-competent CHO cells, as target cells). In FIGS. 6 and 8, E:S=1:0 means the amount ratio of applied expression vector to applied silencing vector is 1:0, E:S=1:1 means the amount ratio of applied expression vector to applied silencing vector is 1:1, and E:S=1:5 means the amount ratio of applied expression vector to applied silencing vector is 1:5. The first two digits of the numeric symbols 10-2, . . . , 11-16, . . . , 15-15, etc in FIG. 6 represent the amount ratio of applied expression vector to applied silencing vector, and the number following the first two digits represent the ID of a tested cell clone (or surviving clone). Similarly, the first two digits of the numeric symbols 10-1, . . . , 11-6, . . . , 15-16, etc in FIG. 8 represent the amount ratio of applied expression vector to applied silencing vector, and the number following the first two digits represent the ID of a tested cell (or surviving clone). In FIGS. 6 and 8, the upper diagrams are for illustrating the result of tests where MTX=0, the middle diagrams are for illustrating the result of tests where MTX=1 μM, and the lower diagrams are for illustrating the result of tests where MTX=5 μM; the vertical axis of each diagram means the relative expression of EGFP protein; the horizontal axis of each diagram is marked by numeric symbols 10-2, . . . , 11-6, . . . , 15-17 or 10-1, . . . , 11-6, . . . , 15-16 aforementioned. It can be seen from FIGS. 6 and 8 the EGFP protein expression resulting from the application of the silencing vector according to the present invention is significantly higher (more than twice) in contrast to those with no-use of silencing vector, regardless of which type of cells are adopted as target cells. The freedom of choosing any type of CHO cells as target cells to enhance protein production according to the present invention, results from the feature of reducing not only the expression of exogenous protein (the protein encoded by the expression vector) but also the expression of endogenous protein (the protein of the target cells). In addition to the freedom of choosing any type of CHO cells as target cells, better stability and shorter time span of producing protein are also among the advantages provided by the feature of the present invention.

Silencing Vector Targeting DHFR Protein (or dhfr Gene) to Improve the Selection of Stable Producer Clones of CHO/dhfr- Cells Through Stepwise MTX Selection

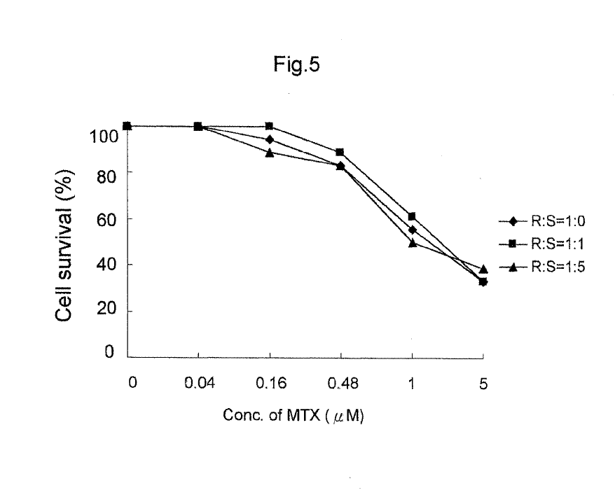

The amplification of the targeted DHFR protein in CHO/dhfr- cells can be obtained by stepwise MTX selection. The expression vector (pEGFP-DHFR) and the silencing vector psd2 (pCMVen-sd2 without CMV enhancer) were mixed in various ratios (R:S=1:0, 1:1, and 1:5); transfected to CHO/dhfr- cells, and then underwent G418 and Zeocin selection, to study the effects of dhfr-targeted RNA silencing vectors on target gene amplification in CHO/dhfr- cells through stepwise MTX selection. Approximately 80 cell clones were obtained from the single-cell cloning of G418/Zeocin-resistant pool cells, and a total of 19 cell clones finally survived the stepwise MTX selection. The use of psd2 silencing vectors did not influence the cell survival rates of CHO/dhfr- cells during stepwise-increased MTX selection (FIG. 5). The stepwise MTX selection process in the CHO cell culture significantly enhanced EGFP expression, as presented in these 19 stable clones at 0 μM, 1 μM and 5 μM MTX (FIG. 6). The use of the sd2 silencing vector resulted in the selection of three high-producer cell clones, 11-15, 11-16 and 15-15, which exhibited an EGFP expression that was over 100% more than those of two high producer clones, 10-9 and 10-10, selected without the use of silencing vector (FIG. 6). Quantitative PCR was further used to determine the numbers of co-amplified gene copies of five high-producing stable clones of CHO/dhfr- cells. Without the use of psd2 silencing vectors, the number of DHFR protein copies were 33.0±4.8 for clone 10-9 and 15.0±2.9 for clone 10-10 at 0 μM MTX, increasing to 233.3±16.3 for clone 10-9 and 458.6±24.8 for clone 10-10 at 5 μM MTX. Stable clones obtained using psd2 silencing vector yielded the number of gene copies equal to 5.1±0.6 for clone 11-15, 2.2±0.1 for clone 11-16, and 1.6±0.3 for clone 15-15 at 0 μM MTX, increasing to 387.1±22.6 for clone 11-15, 442.1±31.5 for clone 11-16 and 240.5±29.2 for clone 15-15 at 5 μM MTX. The results indicated that these high-producing stable clones formed with and without the use of silencing vector for stepwise MTX selection yielded similar numbers of copies ranging from 200 to 400 at 5 μM MTX. Therefore, the use of sd2 silencing vector improves the selecting of stable cell clones with enhanced target gene expression but not the number of co-amplified gene copies by stepwise MTX selection.

Silencing Vector Targeting dhfr Gene (or DHFR Protein) to Improve the Selection of Stable Producer Clones of Normal CHO Cells (CHO-K1 Cells) Through Stepwise MTX Selection

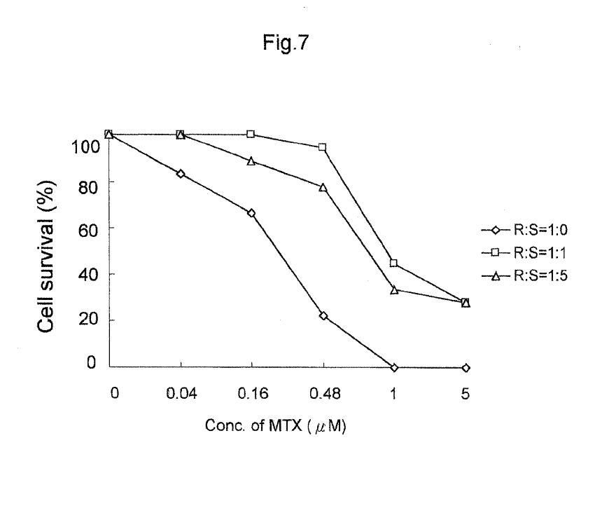

CHO-K1 cells is generally subject to lower frequency of obtaining stable producer cell clones by DHFR/MTX gene amplification since the endogenous DHFR protein in CHO-K1 cells can resist MTX selection. The results reveal that CHO-K1 cells transfected with 1 μg and 1.5 μg of the pCMVen-sd2 silencing vector reduced the amount of endogenous dhfr RNA transcripts by 40%-50% and the endogenous DHFR expression by 70%-80% (data not shown). Whether or not the use of sd2 silencing vector increased the dhfr-directed gene amplification in CHO-K1 cells through stepwise MTX selection was investigated. The sd2 silencing vector was mixed with the expression vector pEGFP-DHFR in various ratios (R:S=1:0, 1:1 and 1:5) and co-transfected into CHO-K1 cells, before undergoing stepwise MTX selection as previously performed in CHO/dhFr- cells. As expected, stable producer CHO-K1 cell clones obtained without the use of sd2 silencing vector did not survive the stepwise MTX selection at 0.48 μM (FIG. 7). Around 20% CHO-K1 stable producer cell clones survived at MTX=5 μM (FIG. 7). The CHO-K1 stable clones (11-6, 11-7, 11-8, 15-1 and 15-16) exhibited enhanced EGFP expression as MTX increased to 1 μM and 5 μM (FIG. 8).

Stability of Stable Producer CHO/dhfr- Cells (dhfr Gene Deficient CHO Cells) and CHO-K1 Cells (Normal CHO Cells) in MTX-Free Medium

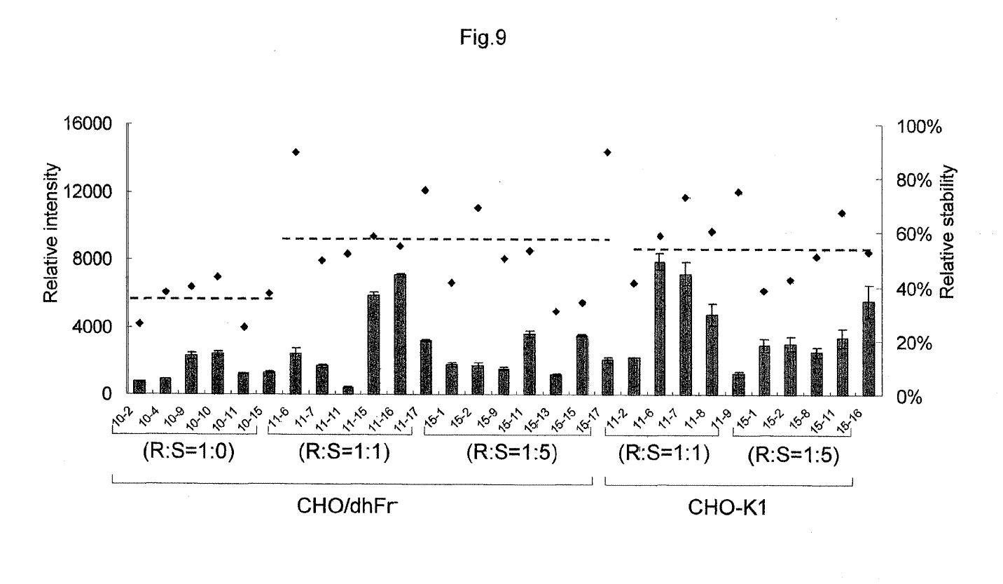

After MTX/dhfr gene amplification process, stable producer cell clones obtained from CHO/dhFr- and CHO-K1 cells were grown in the MTX-free medium for two weeks to elucidate the effect of sd2 silencing vectors on the stability of EGFP expression in stable producer CHO cells. The results indicated that EGFP expression was reduced in CHO/dhfr- and CHO-K1 stable producer cell clones by 30% to 90% in MTX-free medium (FIG. 9). However, with the use of sd2 silencing vectors, on average, the relative stability of EGFP expression in CHO/dhfr- stable clones was 63.32%, and in CHO-K1 stable clones was 56.76%, which values compare to 34.84% in CHO/dhfr- stable clones obtained without the use of sd2 silencing vector (R:S=1:0, FIG. 9). Notably, the stable producer cell clones CHO/dhfr- -11-15, CHO/dhfr- -11-16, CHO/dhfr- -15-17, CHO-K1-11-6, CHO-K1-11-7 and CHO-K1-15-16 had a relative stability of over 53% EGFP expression. These results indicated that the use of sd2 silencing vector also increased the stability of stable producer CHO cells clones for recombinant DNA expression.

Applications of Silencing Vector Targeting DHFR Protein (or dhfr Gene) for Enhancing Secretory IgG Expression in Stable Producer CHO/dhfr-Cells (dhfr Gene Deficient CHO Cells) and CHO-K1 Cells (Normal CHO Cells)

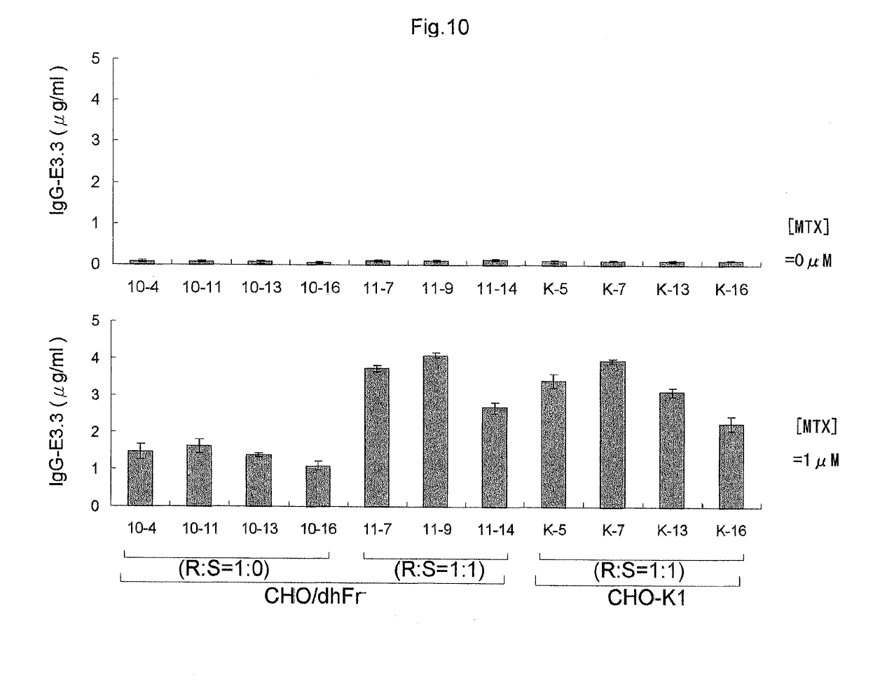

Silencing vector sd2 was co-transfected with the IgG1-E3.3 expression vector into target cells (CHO/dhfr- cells and CHO-K1 cells) at R:S ratios of 1:0 and 1:1 (FIG. 10). The IgG1-E3.3 expression vector encodes mouse DHFR protein and heavy and light chains of a chimeric IgG1-E3.3 targeted on domain III of the Japanese encephalitis virus envelope protein. The silencing vector sd2 is capable of reducing the expressions of both exogenous protein (mouse DHFR protein) and endogenous protein (the protein of the target cells). Selection of more stable producer cell clones with higher productivity can be achieved by the application of the silencing vector, and the advantage can be further expanded by combining stepwise MTX selection. Approximately 80 single cell clones were chosen from the pool of each transformant and 16 highly expressed clones were obtained throughout stepwise MTX selection (0.04 μM→0.16 μM→0.48 μM→1.0 μM). As shown in FIG. 10, the secretion of IgG-E3.3 in these surviving cell clones increased with the MTX concentration from 0 μM to 1 μM. The use of sd2 silencing vector resulted in the selection of five high producer cell clones, 11-7, 11-9, K-5, K-7 and K-13, which exhibited a secretory IgG expression that is more than 100% higher than that of the producer clones selected without a silencing vector (FIG. 10). The yields obtained in high-producing clones using sd2 silencing vector were 8.9-13.6 pg/cell/day (CHO/dhfr- cells) and 7.5-13.2 pg/cell/day (CHO-K1 cells), compared to 3.6-5.4 pg/cell/day (CHO/dhfr- cells without silencing vector).

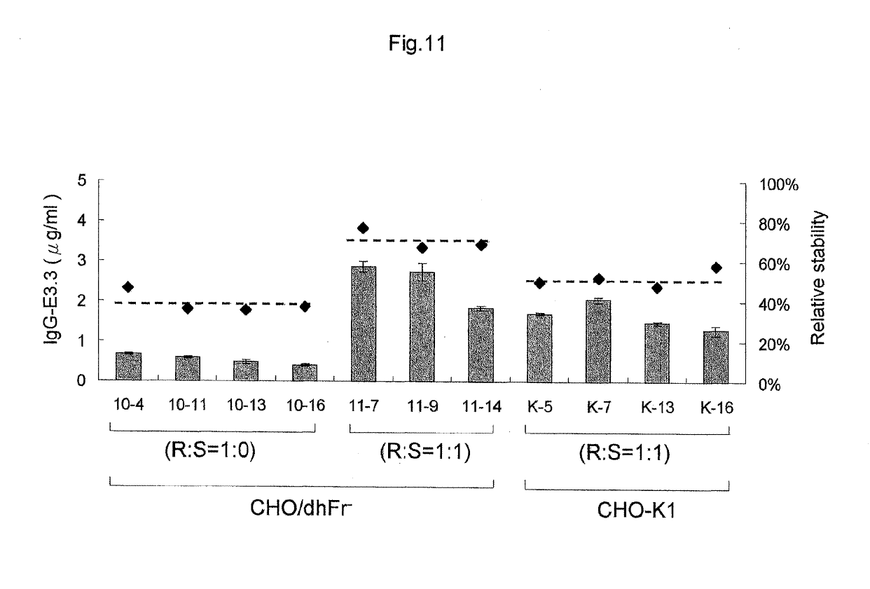

All stable producer cell clones obtained were also grown in the MTX-free medium to elucidate the effect of sd2 silencing vector on the stability of IgG expression in stable producer clones. The results indicated that secretory IgG expression was reduced in stable producer cell clones by 30% to 80% in MTX-free medium (FIG. 11). However, with the use of sd2 silencing vector, on average, the relative stability of IgG expression in CHO/dhfr- stable clones was 70.82%, and in CHO-K1 stable clones was 51.70%, which values compare to 39.03% in CHO/dhfr- stable clones obtained without the use of sd2 silencing vector (FIG. 11). These results further demonstrated that the sd2 silencing vector enhanced the expression of secretory IgG in stable clone through MTX gene amplification and the stability of stable producer CHO cells clones for IgG expression.

### Remarks

Among the advantages of using the silencing vectors provided according to the present invention to enhance protein production (through forming shRNA targeting dhfr sequence), are expression reduction of both exogenous DHFR protein and endogenous DHFR protein, powerful inducing capability, high transfection efficiency, low cytotoxicity, and the dhfr-directed target gene co-amplification throughout stepwise MTX selection. According to the present invention, shRNA silencing vectors are respectively constructed on the basis of segments sd1, sd2, sd3 targeting the conserved sequences of the mouse and Chinese hamster DHFR proteins. Results demonstrates that the silencing vectors targeting DHFR protein, not only induce effective shRNA against the DHFR protein transcripts, but also yield a permanent integration of a silencing cassette, resulting in co-amplification of the dhfr-directed target gene throughout stepwise MTX selection to increase the strength of RNAi specific to the DHFR protein transcripts.

The dhfr-directed target gene amplification can be expanded by a procedure of stepwise increasing MTX concentration corresponding to the increased DHFR expression for subsequent cell growth. As the amplification of the endogenous DHFR protein inevitably results in a low frequency of transfection and gene amplification, no prior art has ever been known to choose normal CHO cells as target cells for producing desired protein. As demonstrated according to the experiments disclosed herein, the reduced, but still existent, expression of endogenous DHFR protein in CHO-K1 cells suffices for generating a highly producing stable cell line by gene amplification. The knockdown of endogenous DHFR protein expression in CHO-K1 cells in this paper resembles that in the cellular environment of the CHO/dhfr- cells, and this down-regulation is sufficient for enhancing amplification of desired protein. Therefore, the silencing cassette in psd2 that is specific to both exogenous (mouse) and endogenous (Chinese hamster) DHFR proteins was co-transfected into CHO-K1 cells, and similar productivity (as determined by comparing FIGS. 6 and 8) and similar efficiency of transfection (FIG. 5) and similar production stability (FIG. 9) were achieved. There is potential the co-amplification in CHO-K1 cells mediated by the silencing vector, can be applied to other dhfr- competent cell lines. This result also suggests the potential of using other well-characterized cell lines that contain an unstable karyotype to produce therapeutic recombinant proteins through gene amplification.

The stability of selected producer cell clones is also important for CHO cell expression of recombinant proteins. Unstable CHO cell karyotypes formed by translocation and homogenous recombination usually result in decrease of target gene expression, particularly in cell cultures with MTX removal. We observed a 60-65% drop in the EGFP and secretory IgG expression in stable producer clones of CHO/dhfr- cells during the first two weeks without silencing vector (FIGS. 9 and 11). Notably, the dhfr-targeted sd2 silencing vector retains 13-36% higher stability in stable producer clones of CHO/dhfr- and CHO-K1 cells with MTX removal. This difference arises presumably because RNAi can suppress the generation of some redundant DHFR at a high concentration of MTX under culturing conditions without selective pressure, so the number of lost copies of the gene is less than that in the absence of the silencing vector during a two-week culture. The integration and subsequent amplification of the exogenous gene in genome are known to be critical to the long-term stability of the culture. The integrated location of the host chromosome and epigenetic repression also influence the stability. Given the factors described above, the position effect probably explains the instability in subclones for a 60-generation culture (data not shown).

In conclusion, the scheme of combining the dhfr silencing vector with the transgene-expressing vector according to the present invention, can enhance expression of desired protein regardless of the type of target cells (i.e., CHO/dhfr- cells and/or CHO-K1 cells can be used as target cells), and the enhancement can be expanded through stepwise gene amplification. Strongly expressed cell clones in CHO/dhfr- and CHO-K1 cells with equally efficient stable transfection can be obtained. As can be seen from the experiments with application of the silencing vectors provided according to the present invention, production of secretory protein can be enhanced even without MTX application, and the enhancement can be upgraded with MTX application. Stability of production of secretory protein in the absence of MTX, is also increased by the application of the silencing vectors provided according to the present invention. There is potential the application of the silencing vectors provided according to the present invention may lead up to new means of investigating gene amplification in other wild-type (dhfr-competent) cell lines for promoting/enhancing expression of recombinant proteins.

### DRAWING LEGENDS

FIGS. 1A, 1B

FIG. 1A shows sequence alignment analysis of the DHFR protein and three selected segments respectively for constructing different DNA fragments each for a silencing vector. The number of DHFR proteins of mouse (SEQ ID NO: 1) and Chinese hamster (SEQ ID NO: 2) are shown and the consensus nucleotides symbolized as dash. Three segments (respectively represented by sd1 (SEQ ID NO: 3), sd2 (SEQ ID NO: 4), and sd3 (SEQ ID NO: 5)) of 19 consensus nucleotides behind two adenines (A), each selected for designing a shRNA, was respectively indicated in three rectangles. FIG. 1B shows what are included in the double-stranded RNAs supposed to be transcripted in a target cell to which the silence vector is applied or transfected, wherein the double-stranded RNA marked with SEQ ID NO: 6 is designed according to sd1, the one marked with SEQ ID NO: 7 is designed according to sd2, and the one marked with SEQ ID NO: 8 is designed according to sd3.

FIG. 2

Schematic drawing of reporting and expression vectors. The reporting vector (pDHFR/EGFP) containing DHFR/EGFP fusion protein and expression vector (pEGFP-DHFR) including the egfp and dhfr sequences under two separate promoters: CMV and SV40, were shown here. EGFP, enhanced green fluorescent protein; IRES, internal ribosome entry site from encephalomyocarditis virus; Zeo, zeocin resistance gene; BpA, bovine growth hormone polyadenylation signal; SV40, SV40 early promoter; dhfr, dihydrofolate reductase gene of mouse; SpA, SV40 early polyadenylation signal.

FIG. 3

Screening of silencing vectors to DHFR protein. One μg of reporting vector (pDHFR-EGFP) and two μg of each silencing (pCMVen-sd1, pCMVen-sd2, and pCMVen-sd3) or control (pCMVen, without shRNA) vector were co-transfected into CHO/dhfr- cells to silence the DHFR/EGFP fusion protein; the fluorescence was determined using a Wallac Victor² Multicounter and the relative fluorescence were indicated. The experiments were performed in triplicate; the means (±standard deviation) of EGFP fluorescence intensity are presented.

FIGS. 4A and 4B

RNA silencing to DHFR protein in CHO/dhfr- cells. CHO/dhfr- cells were transfected with increasing amounts of pCMVen-sd2 and the same amount of expression vector (pEGFP-DHFR), which expresses mouse DHFR protein and was used for the following experiments. Fifty hours following transfection, the cells were harvested and separated on 12% SDS-PAGE. Immunoblotting with DHFR-specific antibody was performed and the bands that correspond to the DHFR protein and an internal control (GAPDH) are indicated. The ratio of DHFR expression to GAPDH expression was determined using Gel-Pro 3.1, as shown in FIG. 4A. The amount of dhfr transcript from transfected cells was also measured by quantitative real-time RT-PCR and 18s rRNA is used as an internal control for normalization, as shown in FIG. 4B.

FIG. 5

Survival rate of transfected CHO/dhfr- cells during stepwise MTX selection/amplification. Survival rate of clones from CHO/dhfr- with different ratio of expression vectors and silencing vectors were calculated. The data are shown as the percentage of clones surviving at the indicated concentration of MTX relative to the original clones. The expression vectors (pEGFP-DHFR, R) and silencing vectors (psd2, S) vectors in different ratios (1:0, 1:1, and 1:5) were indicated.

FIG. 6

Co-transfection of silencing vector increases protein expression in CHO/dhfr- cells. The linearized vectors were transfected into CHO/dhfr- cells, and 18 colonies that most strongly expressed EGFP were isolated. During the stepwise increase in the concentration of MTX in the growth medium, the fluorescent intensity of EGFP expressed in survival colonies at MTX concentrations of 0 (A), 1.0 (B) and 5.0 μM (C) were measured. The expression vectors (pEGFP-DHFR, E) and silencing vectors (psd2, S) vectors in different ratios (1:0, 1:1, and 1:5) were introduced into CHO/dhfr- cells as indicated.

FIG. 7

Survival rate of transfected CHO-K1 cells (dhfr-competent cells) during stepwise MTX selection/amplification. Survival rate of clones from CHO-K1 with different ratio of expression vectors and silencing vectors were calculated. The data are shown as the percentage of clones surviving at the indicated concentration of MTX relative to the original clones. The expression vectors (pEGFP-DHFR, E) and silencing vectors (psd2, S) vectors in different ratios (1:0, 1:1, and 1:5) were indicated.

FIG. 8

Co-transfection of silencing vector increases protein expression in dhfr-competent CHO-K1 cells. The linearized vectors were transfected into CHO-K1 cells, and 18 colonies that expressed the highest EGFP levels were isolated. After the MTX concentration was stepwise increased in the growth medium, the fluorescent intensity of EGFP was measured (A, B, and C). The expression vectors (pEGFP-DHFR, E) and silencing vectors (psd2, S) vectors in different ratios (1:0, 1:1, and 1:5) were introduced into CHO/dhfr- cells, as indicated. Transformants that contained pEGFP-DHFR vector only could not endure an MTX pressure of over 1.0 μM, so no CKO-K1 (1:0) clone is presented here.

FIG. 9

Stability of recombinant proteins produced in CHO cells. Fluorescence of EGFP expressed in transformants under culture conditions without selection pressure (MTX). After stepwise amplification in 5 μM MTX, each surviving clone was transferred to the fresh growth medium without MTX, and subcultured five times in 15 days. The figure presents the final fluorescence and the percentage to the original value (MTX=5 μM, black diamond) for each clone.

FIG. 10

The IgG secretion of the stable clones. Three-days after inoculation, the concentration of antibody in the supernatant of clones expressing E3.3 at MTX concentrations of 0 and 1.0 μM were determined by ELISA. The IgG-expression vectors (E) expressing IgG and silencing vectors (psd2, S) vectors in different ratios (1:0, 1:1, and 1:5) were indicated. The specific productions (pg/cell/day) were calculated and shown here.

FIG. 11

Stability of secretory IgG produced in CHO cells. Concentration of IgG expressed in transformants under culture conditions without selection pressure (MTX). Each surviving clone in 1 μM MTX was transferred to the fresh growth medium without MTX and subcultured five times in 15 days. The figure presents the final concentration and the percentage to the original value (MTX=1 μM, black diamond) for each clone.

While the invention has been described in terms of what are presently considered to be the most practical and preferred embodiments, it shall be understood that the invention is not limited to the disclosed embodiment. On the contrary, any modifications or similar arrangements shall be deemed covered by the spirit of the present invention.

What is claimed is:

1. Material for applying to at least a CHO cell for enhancing production of secretory protein, comprising:
an expression vector including a first sequence encoding DHFR protein of a species of mammal and a second sequence encoding the secretory protein; and
a silencing vector including a DNA fragment for inducing a RNA interference capable of reducing expression of the DHFR protein of said species of mammal and expression of the DHFR protein of said CHO cell after said material is applied to said CHO cell.

2. The material according to claim 1 wherein said species of mammal is mouse.

3. The material according to claim 1 wherein said DNA fragment is SEQ ID NO. 4.

4. The material according to claim 1 wherein said secretory protein is antibody.


SEQUENCE LISTING (8 sequences)

SEQ ID NO: 1 - DNA, 564 bp, Mus musculus
atggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaa

SEQ ID NO: 2 - DNA, 564 bp, Cricetulus griseus
atggttcgaccgctgaactgcatcgtcgccgtgtcccagaatatgggcatcggcaagaacggagaccttccctggccaatgctcaggaacgaattcaagtacttccaaagaatgaccaccacctcctcagtggaaggtaaacagaacttggtgattatgggccggaaaacctggttctccattcctgagaagaatcgacctttaaaggacagaattaatatagttctcagtagagagctcaaggaaccaccacaaggagctcattttcttgccaaaagtctggacaatgccttaaaacttattgaacaaccagagttagcagataaagtcgacatggtttggatagttggaggcagttccgtttacaaggaagccatgaatcagccaggccatctcagactctttgtgacaaggatcatgcaggaatttgaaactgacacgttcttcccagaaattgatttggagaaatataaacttctcccagagtacccaagggtccttcctgaagtccaagaggaaaaaggcatcaagtataaatttgaagtctatgagaagaaaggctaa

SEQ ID NO: 3 - DNA, 19 bp, Mus musculus <s300> Wu, Suh-Chin and Hong, Willy W.L. A Novel RNA Silencing Vector to Improve Antigene Expression and Vaccine 25 20 4103-4111 2007-05-16
ggatcatgcaggaatttga

SEQ ID NO: 4 - DNA, 19 bp, Mus musculus <s300> Wu, Suh-Chin and Hong, Willy W.L. A Novel RNA Silencing Vector to Improve Antigene Expression and Vaccine 25 20 4013-4111 2007-05-16
gtacttccaaagaatgacc

SEQ ID NO: 5 - DNA, 19 bp, Mus musculus <s300> Wu, Suh-Chin and Hong, Willy W.L. A Novel RNA Silencing Vector to Improve Antigene Expression and Vaccine 25 20 4103-4111 2007-05-16
ggacagaattaatatagtt

SEQ ID NO: 6 - DNA, 47 bp, Artificial sequence
  chemically synthesized
ggatcatgcaggaatttgacttaagctcaaattcctgcatgatcctt

SEQ ID NO: 7 - DNA, 47 bp, Artificial sequence
  chemically synthesized
gtacttccaaagaatgacccttaagcggtcattctttggaagtactt

SEQ ID NO: 8 - DNA, 47 bp, Artificial sequence
  chemically synthesized
ggacagaattaatatagttcttaagcaactatattaattctgtcctt